(19) United States
(12) Patent Application Publication
(10) Pub. No.: US 2019/0009060 A1
(43) Pub. Date: Jan. 10, 2019

(54) TRACKING UNIT, ENDOVASCULAR DEVICE WITH FLUOROLESS AND WIRELESS TRACKING UNIT, COMPATIBLE IMAGING SYSTEM, AND RELATED METHODS

(71) Applicant: Paula Eboli, Spokane, WA (US)

(72) Inventor: Paula Eboli, Spokane, WA (US)

(21) Appl. No.: 16/024,524

(22) Filed: Jun. 29, 2018

Related U.S. Application Data
(60) Provisional application No. 62/526,934, filed on Jun. 29, 2017.

(51) Int. Cl. A61M 25/01; A61B 34/20; A61M 25/06; A61B 5/06

(52) U.S. Cl. CPC A61M 25/0127; A61B 34/20; G01R 33/286; A61B 5/065; A61B 2034/2051; A61M 25/0662

(57) ABSTRACT

An embodiment of an apparatus includes first and second tracking units configured for mounting to an endovascular device, and respectively configured to generate a first magnetic field along a first dimension and a second magnetic field along a second dimension that is approximately orthogonal to the first dimension. And an embodiment of an endovascular device includes a body and first and second tracking units. The body is configured for insertion into a lifeform. The first tracking unit is disposed at a first location of the body and includes a first coil configured to generate a first signal related to the first location in response to a first magnetic field. And the second tracking unit is disposed at a second location of the body and includes a second coil configured to generate a second signal related to the second location in response to a second magnetic field.

### CROSS-RELATED APPLICATIONS

This application claims benefit of priority to the following U.S. patent application, which is incorporated by reference: U.S. Provisional Patent Application Ser. No. 62/526,934 entitled “ENDOVASCULAR DEVICE WITH FLUOROLESS AND WIRELESS TRACKING POINT, COMPATIBLE IMAGING SYSTEM, AND RELATED METHODS,” filed 29 Jun. 2017.

### SUMMARY

Conventional imaging systems for endovascular procedures currently suffer from various deficiencies.

For example, a medical professional who performs an endovascular procedure may be exposed to dangerous high-frequency, high-power, radiation, e.g., x-rays, generated by a conventional imaging system, where such radiation can cause severe health conditions.

One way for a medical profession to protect himself/herself is to wear protective gear.

But the protective gear typically presents its own set of problems.

For example, because the protective gear is typically bulky and heavy, wearing protective gear can hinder the medical professional's movements, and this his/her ability to perform a procedure like an endovascular procedure. And in some cases, the gear is so heavy that a medical professional cannot bear the entire weight of the gear, at least not while performing a medical procedure such as an endovascular procedure. In this latter situation, although one or more cables can be used to support, most, if not all, of the weight of the gear from the ceiling, the installing of reinforced cable connectors in the ceiling is expensive and time consuming, one or more people in addition to the medical professional may be needed to hang the gear from the cables and to assist the medical professional in donning the gear, and the cables may restrict the movement of the medical professional.

Furthermore, the protective gear may leave some areas of the medical professional's body exposed.

An endovascular device that solves one or more of the above problems (and possibly solves other problems) uses low-frequency, low-power, signals to pinpoint, or otherwise locate, important areas during an endovascular procedure. Such an endovascular device may use less energy, and may be safer for medical professionals performing a procedure, than a conventional imaging system.

For example, in an embodiment, an apparatus for use with an endovascular device includes first and second tracking units. The first tracking unit is configured for mounting to the endovascular device and to generate a first magnetic field along a first dimension. And a second tracking unit is configured for mounting to the endovascular device and to generate a second magnetic field along a second dimension that is approximately orthogonal to the first dimension.

In another embodiment, an endovascular device includes a body and first and second tracking units. The body is configured for insertion into a lifeform, such as a human or other animal. The first tracking unit is disposed at a first location of the body and includes a first coil configured to generate a first signal in response to a first magnetic field, the first signal related to the first location. And the second tracking unit is disposed at a second location of the body and includes a second coil configured to generate a second signal in response to a second magnetic field, the second signal related to the second location.

### BRIEF DESCRIPTION OF THE DRAWINGS

FIG. 1 is an image of an endovascular device, such as a catheter or guide wire, disposed within a blood vessel of a body.

FIGS. 2A and 2B are images of respective medical professionals wearing protective gear suspended from the ceiling while performing respective endovascular procedures using conventional fluoroscopy.

FIG. 3 is an isometric view of an endovascular device for insertion into a body, the device including one or more tracking points (also called “tracking units”) along the length of the device, according to an embodiment.

FIG. 4 is an isometric side view, with portions broken away, of the endovascular device of FIG. 3 with the tracking points situated at the end of the device, according to an embodiment.

FIG. 5 is an isometric side view of the endovascular device of FIG. 3 with the tracking points situated along the outer circumference of the device, according to an embodiment.

FIG. 6 is an isometric side view of the endovascular device of FIG. 3 with the tracking points situated in alignment at the end of the device, according to an embodiment.

FIG. 7 is an isometric side view of the endovascular device of FIG. 6, and illustrates the magnetic flux generated by an activated tracking point, according to an embodiment.

FIG. 8 is an isometric view of a tracking point of FIG. 7, and shows a magnetic field generated by the tracking point while active, according to an embodiment.

FIG. 9 is an electrical circuit diagram of a tracking point of FIGS. 3-8, according to an embodiment.

FIG. 10 is a plot of a signal generated by the electrical circuit of FIG. 9 during tracking-point excitation and tracking-point tracking periods, according to an embodiment.

FIG. 11 is an isometric view of an excitation coil and a tracking coil of a tracking point of FIGS. 3-8, according to an embodiment.

FIG. 12 is an isometric view of a coil assembly of a tracking point of FIGS. 3-8, the coil assembly including spatially orthogonal coils, according to an embodiment.

FIG. 13 is a perspective view of an endovascular imaging system that is configured to excite, and to sense a signal generated by, a tracking point of FIGS. 3-8, according to an embodiment.

FIG. 14 is an image of an aneurismal blood vessel inside of a body and of the endovascular device of FIGS. 3-6 inside of the blood vessel, according to an embodiment.

FIG. 15 is an image of an arterial-venous malformation (AVM), and of surrounding blood vessels, according to an embodiment.

FIG. 16 is a view of an endovascular device having electromagnetic antenna coils, according to an embodiment.

FIG. 17 is a perspective view of an endovascular-device-and-tracking system, which includes the endovascular device of FIG. 16 and a tracking subsystem, according to an embodiment.

FIG. 18 is a diagram of circuitry of the endovascular device of FIGS. 16-17, according to an embodiment.

FIG. 19 is a diagram of circuitry of the endovascular device of FIGS. 16-17, according to another embodiment.

### DETAILED DESCRIPTION

In the following description, each value, quantity, or attribute herein preceded by “substantially,” “approximately,” “about,” a form or derivative thereof, or a similar term, encompasses a range that includes the value, quantity, or attribute ±20% of the value, quantity, or attribute, or a range that includes ±20% of a difference between the ends of the range. For example, “approximately 1.0 V” encompasses a voltage 0.8 V≤α≤1.2 V, and “an approximate range of 0.40 V-1.00 V” encompasses a range of 0.28 V-1.12 V. Furthermore, two axes are substantially parallel to one another encompasses an angle of −18°≤α≤+18° between the two axes |90°| is the maximum angular difference between the two axes, ±20% of |90°| is ±18°, and the two axes are parallel to one another when α=0°). Moreover, signals (e.g., voltage, current, magnetic field) are assumed to be functions of time unless otherwise noted.

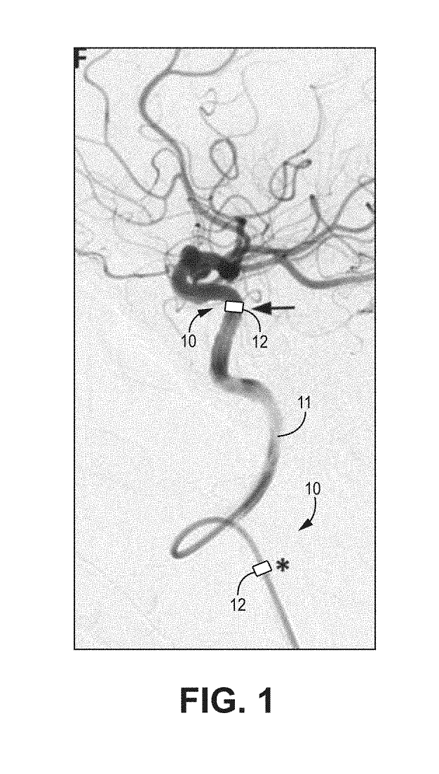

FIG. 1 is an image of an endovascular device 10, such as a catheter or guide wire, disposed within a blood vessel 11 of a body and marked with a radiopaque material at periodically spaced locations 12 so that the device can be imaged with a fluoroscopic device while the device is inside of the blood vessel.

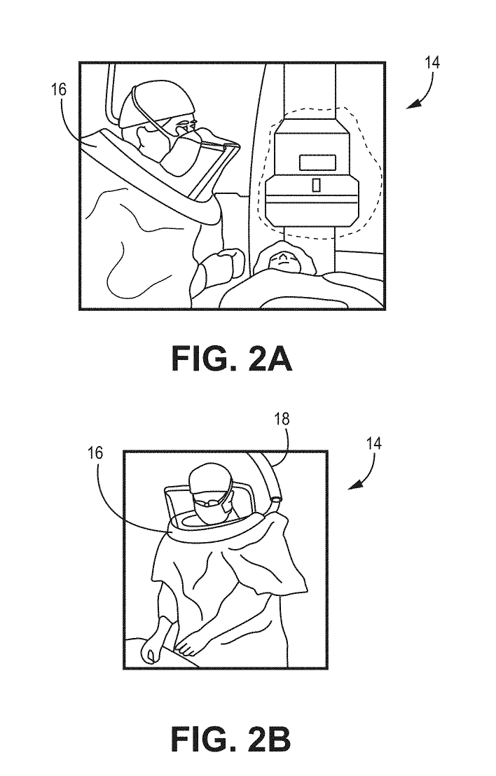

Referring to FIGS. 1-2, while the endovascular device 10 is inside the body, e.g., inside of the blood vessel 11, an imaging system 14, such as a Biplane Fluoro Machine, generates high-energy and high-frequency (short wavelength) radiation (e.g., x-rays) that penetrates the body but is blocked by the radiopaque material.

Consequently, the imaging system 14 can sense shadows in the radiation blocked by the radiopaque material, can determine, in response to the sensed radiation pattern, the locations 12 marked with the fluorescent material, and can generate, on a display screen, one or more images including representations (e.g., dots) of the locations. By viewing the locations 12 within the image of the part (e.g., blood vessel) of the human body on which a doctor (or other medical professional) is performing a procedure, the doctor can determine the current position of the endovascular device 10 within the body, and can visually track the endovascular device, or locations (e.g., an end) thereof, as he/she manipulates the device within the body part.

Still referring to FIGS. 1-2, a problem with the imaging system 14 is that a doctor (or other medical professional) who performs endovascular procedures on a regular basis can be repeatedly exposed to the high-energy radiation generated by the imaging system, and such repeated high-energy-radiation exposure can be harmful to the doctor.

To protect himself/herself from such potentially harmful high-energy-radiation exposure, a doctor can wear protective gear 16 while he/she is performing an endovascular procedure.

But a problem with the protective gear 16 is that it is typically very heavy (e.g., approximately 25 pounds) and it only covers some areas of the body; the areas of the body that the protective gear leaves uncovered may be exposed to radiation.

Furthermore, the protective gear 16 may be so heavy that it is at least partially supported by wires, cables, or other members 18, which extend from a ceiling or other support structure.

But even though such ceiling support may reduce the apparent weight of the protective gear 16 to the doctor, the protective gear still may impede the doctor's freedom of movement, particularly while he/she is performing the endovascular procedure.

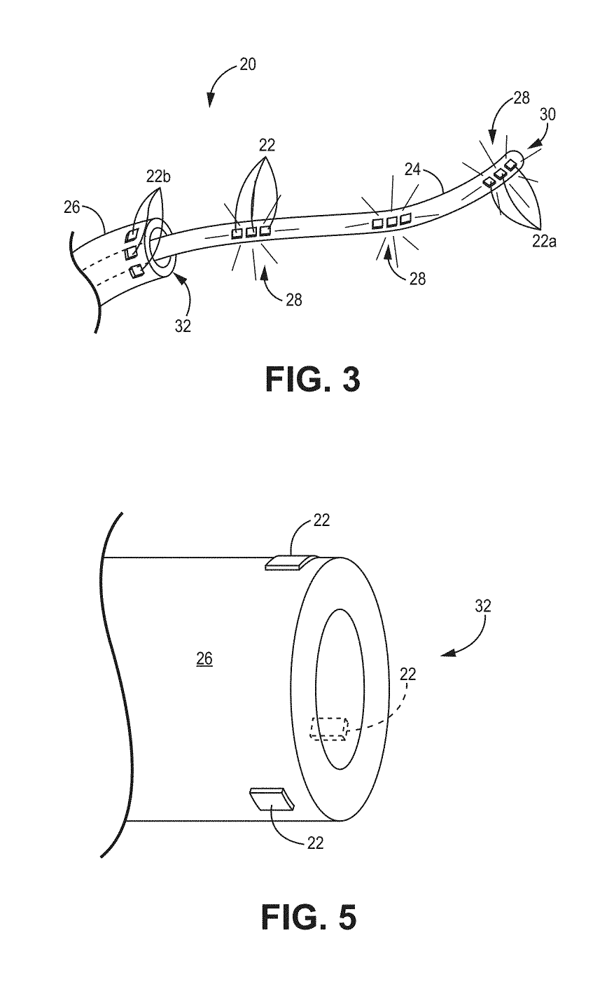

Referring to FIG. 3, an endovascular device 20 includes one or more tracking points (also called “tracking units”) 22, which allow a significant reduction in the level of high-energy, and potentially harmful, radiation to which a doctor is exposed, or even a total elimination of such potentially harmful radiation, without the need for protective gear. Also the tracking points 22 can be configured to allow reducing, and possibly eliminating, the need for iodine contrast for some procedures. Allowing a reduction, or elimination, of iodine contrast can be very important in patients with renal failure, as the use of iodine contrast can exacerbate renal failure in such patients. And using tracking points 22 to indicate of a position of an endovascular device within a body can be achieved using previously obtained vascular images, together with image fusion (e.g., fusing a real-time image of the device within a region of a body with an image of the body's major blood vessels in that region of the body) and virtual-imaging techniques.

As described below, the tracking points 22 are excitable by non-ionizing electromagnetic signals having much lower frequencies, and therefore, having much lower energies than the high-energy radiation (e.g., x-rays) used to excite the fluorescent material described above in conjunction with FIGS. 1-2. Fortunately, even with repeated and unprotected exposure to such lower-energy electromagnetic signals, a doctor suffers little or no adverse effects.

Furthermore, the tracking points 22 are configured to provide a wireless solution to imaging an endovascular device 20 within a body. That is, in an embodiment, no power or signal wires need to be connected to the endovascular device 20 while it is in a blood vessel (or in another part of the body) to excite the tracking points 22; instead, the tracking points are configured to be excited wirelessly, and the magnetic fields generated by the excited tracking points can be sensed wirelessly.

Moreover, the tracking points 22 are small enough for use on small endovascular devices 20 such as, for example, catheters, guide wires, stent placers, stent removers, stent retrievers, stents, and any other type of endovascular device.

As described below, an activated tracking point 22 is configured to act as a magnetic dipole that generates a magnetic field. Unlike a scalar quantity, such as what effectively is the brightness contrast that a radiopaque material generates in the high-energy radiation field, a magnetic field is a vector quantity. Therefore, as described below, for a single-plane image a tracking point 22 can be configured to provide information about a location or movement of an endovascular device 20, information that a radiopaque material does not, or cannot, provide. And even for a biplane image (e.g., two component images of the same volume in approximately orthogonal planes), a tracking point 22 can be configured to provide at least the same level of position and orientation information that a radiopaque material can provide.

Still referring to FIG. 3, in an embodiment, the endovascular device 20 includes a guide wire 24, which is free to slide within a catheter or sheath 26, and the tracking points 22 are located at respective locations 28 along the endovascular device, the locations being suitable for a procedure for which the endovascular device is configured. For example, tracking points 22a are located at, and are configured to indicate, an end 30 of the guide wire 24. Likewise, tracking points 22b are located at, and are configurable to indicate, an end 32 of the catheter 26. The more closely spaced the tracking-point locations 28, the more accurately an imaging device can determine (e.g., interpolate) the locations, shapes, and other parameters of portions of the endovascular device 20 between the tracking-point locations (such interpolation is further described below).

Because, while active, each tracking point 22 generates a vector magnetic field, including two, three, or more tracking points 22 at a location 28 can facilitate tracking the following parameters for the location in two- or three-dimensional space: position, rotational orientation, linear velocity, linear acceleration, rotational velocity, and rotational acceleration (collectively “position and movement information”).

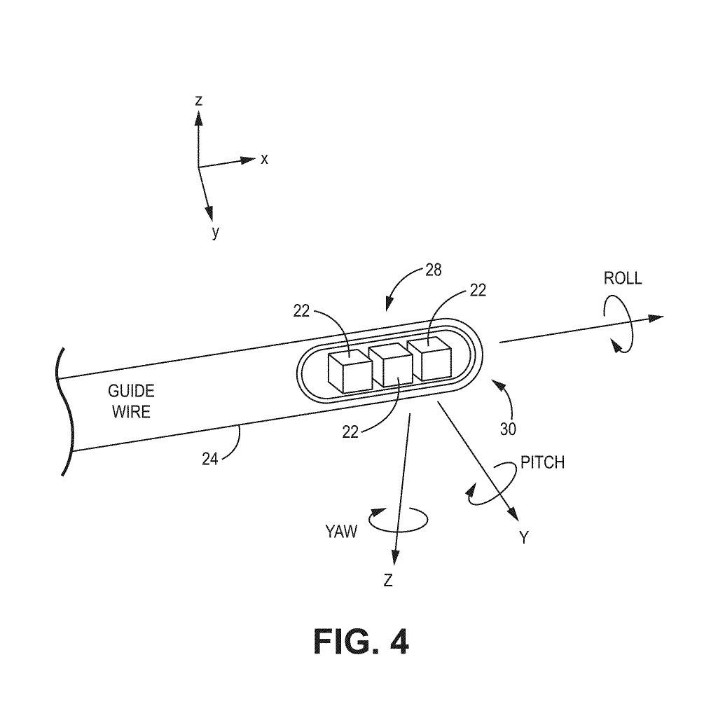

For example, referring to FIG. 4, which is a close-up view of the end 30 of the guide wire 24 of FIG. 3, orienting three tracking points 22 at a location 28 (e.g., guide-wire end 30) such that their magnetic fields are spatially and electrically orthogonal to one another can allow determination of the pitch, yaw, and roll of the guide wire at the location. The magnetic fields can be made spatially orthogonal to one another by aligning a main field line of each tracking point 22 with a respective one of the x, y, and z axes. And the magnetic fields can be made electrically orthogonal to one another by causing each field to have a respective frequency, a respective phase, or a respective amplitude that renders each field electrically orthogonal to one or both of the other fields.

Referring to FIG. 5, multiple tracking points 22 can be spaced apart on an endovascular device 20 (a catheter 26 in FIG. 5) a significant distance from one another yet still provide position and movement information about a location of the endovascular device or of a component thereof. For example, multiple (here three) tracking points 22 can be spaced circumferentially, and approximately equidistantly, around an end 32 of a catheter 26 yet still provide position and movement information regarding the end of the catheter.

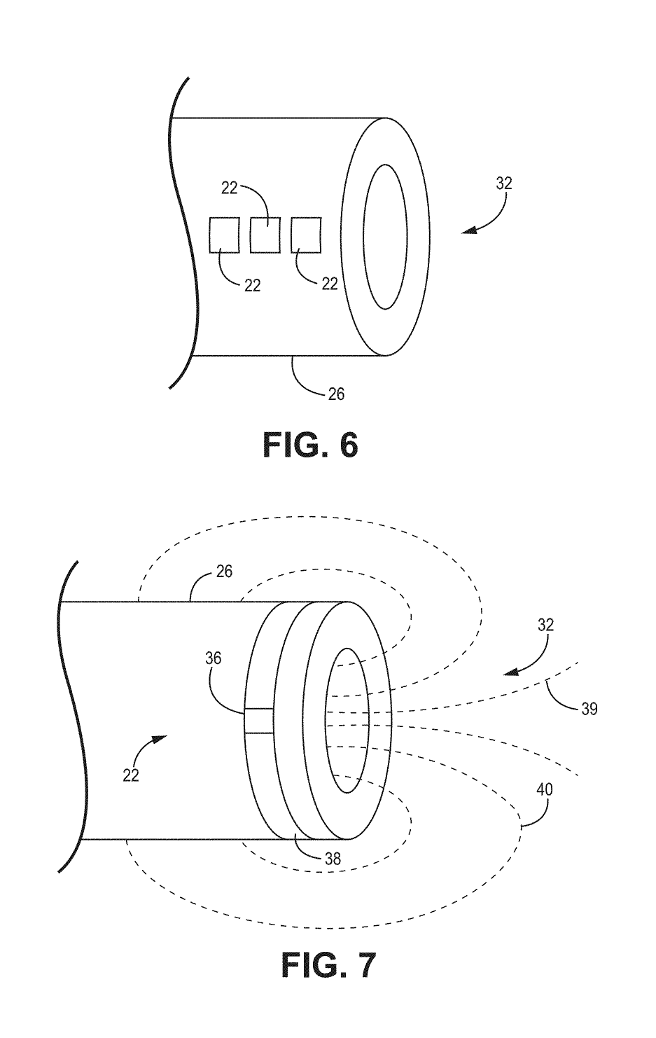

FIG. 6 is a diagram of an alternate arrangement of three tracking points 22 at an end 32 of a catheter 26, according to an embodiment. The tracking points 22 are arranged linearly (i.e., are aligned along a line), and can be oriented so that the magnetic fields they generate are spatially and electrically orthogonal to one another as described above.

FIG. 7 is a diagram of a distributed tracking point 22 disposed at an end 32 of a catheter 26, according to an embodiment. The distributed tracking point 22 includes a capacitor 36 and a conductive coil 38, which forms an inductor, circumferentially wrapped around the end 32 of the catheter 26. While active, the tracking point 22 generates a magnetic field having lines of flux 40 such that a main field line is approximately aligned with, or approximately parallel to, a longitudinal axis 39 of the catheter 26. Operation of a tracking point 22 formed from a capacitor and inductor is described in more detail below.

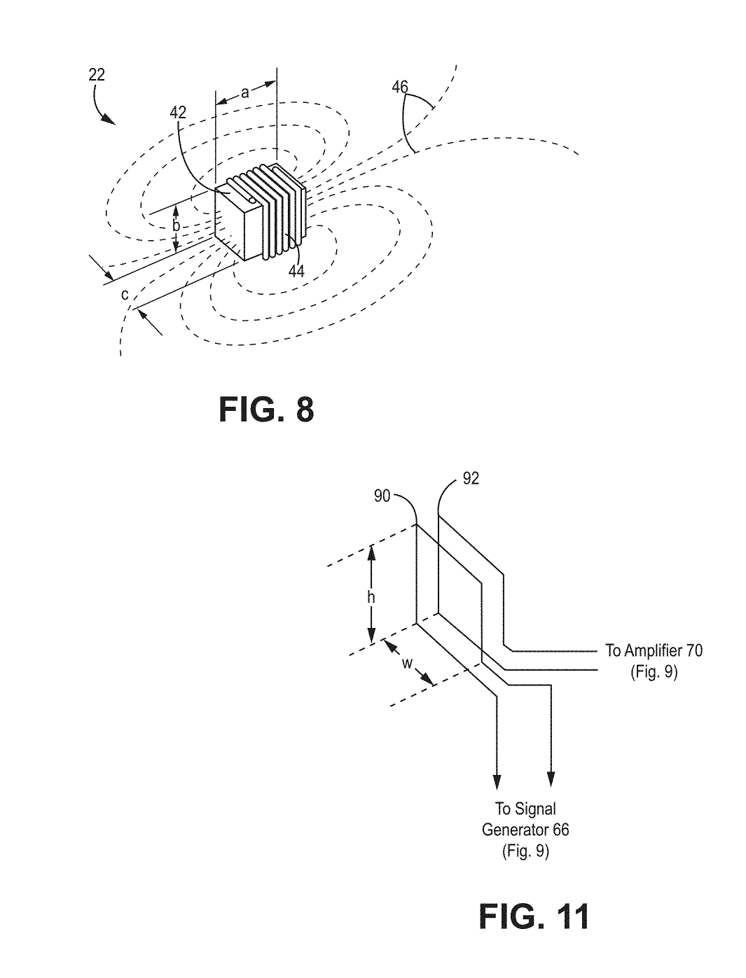

FIG. 8 is a diagram of a tracking point 22, according to another embodiment. The tracking point 22 includes a capacitor 42 (e.g., a ceramic capacitor) and a conductive coil 44, which forms an inductor, wrapped around the capacitor. While active, the tracking point 22 generates a magnetic field having lines of flux 46. Furthermore, the tracking point 22 can have a size and shape that approximates an a×b×c cube, where a, b, and c can be in an approximate range of 0.1 millimeter (mm)≤a, b, c≤1.0 mm. Such a size and shape is sufficiently small for use with an endovascular device 20 configured for insertion into even relatively small blood vessels or other regions of a body. Operation of a tracking point 22 formed from a capacitor and inductor is described in more detail below.

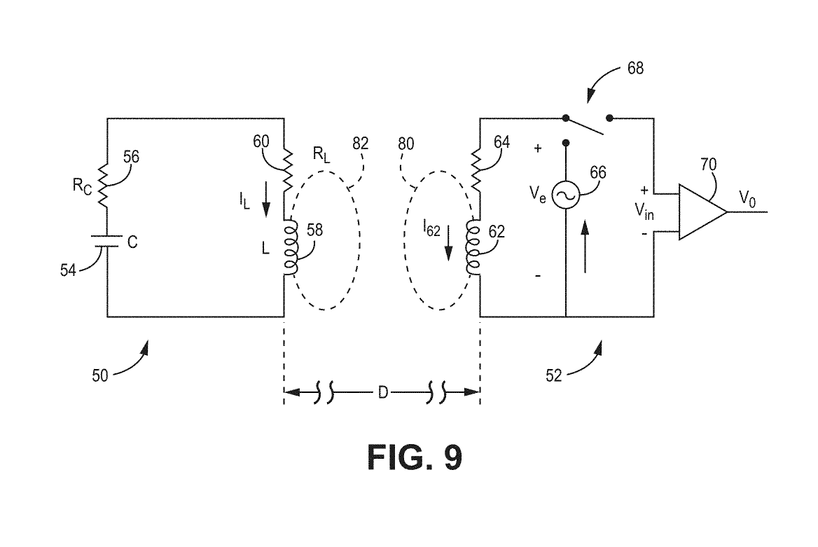

FIG. 9 is a schematic diagram of a tracking-point circuit 50 and a tracking-point-excitation-and-sensing circuit 52, according to an embodiment. A respective instantiation of the tracking-point circuit 50 can be included as part of, or can be formed by, each of the tracking points 22 of FIGS. 3-8.

The tracking-point circuit 50 includes a capacitor 54 having a capacitance C and an equivalent series resistance (ESR) 56 having a resistance R_{C}, and includes an inductor 58 having an inductance L and a DC resistance (DCR) 60 having a resistance R_{L}.

The tracking-point-excitation-and-sensing circuit 52 includes an inductor 62 having a DC resistance (sometimes referred to as “DCR”) 64, a signal source 66, a switch 68 (e.g., a Metal-Oxide-Semiconductor (MOS) transistor or a bipolar transistor), and an amplifier 70 (e.g., an operational amplifier), and can be part of an endovascular imaging system (not shown in FIG. 9).

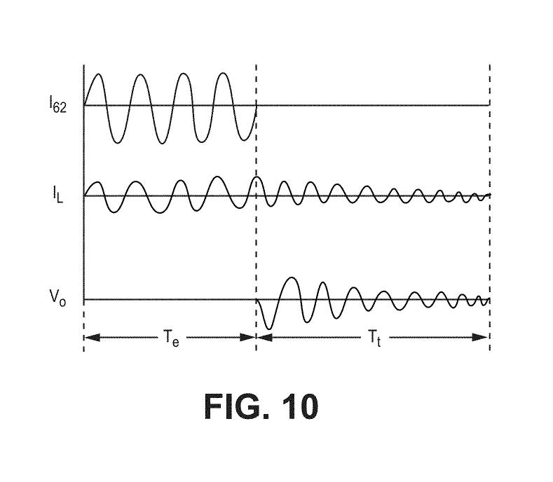

FIG. 10 includes plots versus time of the current I₆₂ through the inductor 62 of FIG. 9, the current I_{L} through the inductor 58 of FIG. 9, and the voltage Vₒ generated by the amplifier 70 of FIG. 9, during an excitation period Tₑ and during a tracking period Tₜ (the tracking-point circuit 50 is excited during the excitation period Tₑ and is active during the tracking period Tₜ).

Referring to FIGS. 9-10, operation of the tracking-point circuit 50 and the tracking-point-exciter-and-sensor circuit of 52 is described, according to an embodiment.

First, the endovascular imaging system, or a human operator, moves the inductor 62 so that it is within the near magnetic field of the inductor 58. For example, assuming that the tracking-point circuit 50 is part of a guide wire that a doctor has inserted into a subject's head, the imaging system or human operator moves the inductor 62 within a distance D from the subject's head, where D is such that the inductor 62 is within the near magnetic field of the inductor 58, and vice-versa. For example, an approximate range of D is 0.0 meters (m)≤D≤1.0 m. Furthermore, although discussed in terms of moving the inductor 62, because the entire exciter circuit 52 is housed within a portion of the endovascular imaging system, typically the system, or a human operator, moves the entire portion of the imaging system such that the inductor 62 is within the range D of the inductor 58.

Next, the endovascular system or human operator causes the switch 68 to couple the signal source 66 across the series combination of the inductor 62 and the resistance 64 (as stated above, the resistance 64 can be the resistance of the winding that forms the inductor 58).

In response to this coupling, the signal source 66 generates an AC excitation voltage Vₑ across the inductor 62 and the resistance 64 to commence the excitation period Tₑ.

The AC voltage Vₑ generates a current I₆₂=Iₑ through the inductor 62, where Iₑ is proportional to Vₑ and inversely proportional to the inductance L₆₂ of the inductor 62 and the resistance R₆₄ of the resistance 64.

Ideally, to promote the most efficient magnetic coupling between the inductors 58 and 62 the frequency fₑ of Vₑ and Iₑ is equal to the resonant frequency f₀½π\sqrt{LC} of the tracking-pointer circuit 50. For example, if L=5.6 micro Henries (μH) and C=0.1 micro Farads (μg), then f₀≈212 Kilohertz (KHz), which is a frequency suitable for transmission through tissues and non-tissue objects inside of a human or other animal body. Typically, a frequency within an approximate range of 0 Hz-1 Megahertz (MHz) is suitable for transmission through tissues and non-tissue objects inside of a human body.

In response to Iₑ, the inductor 62 generates a magnetic field with lines of magnetic flux 80.

The magnetic flux 80 induces, through the inductor 58, an AC excitation current I_{L}=Iₜ having the frequency f₀, even if the frequency fₑ does not equal f₀. That is, the magnetic flux 80 excites the tracking-point circuit 50 by causing an excitation current Iₜ to flow through the capacitor 54, inductor 58, and resistances 56 and 60. During the excitation period Tₑ, the tracking-point circuit 50 operates as a signal-driven tuned resonant circuit.

Therefore, during the excitation period Tₑ, the current Iₑ having a frequency fₑ flows through the inductor 62, and the current Iₜ having a frequency f₀ flows through the inductor 58.

To end the excitation period Tₑ and to commence the tracking period Tₜ, the endovascular imaging system toggles the switch 68 such that it uncouples Vₑ from across the series combination of the inductor 62 and the resistance 64, and couples the series combination of the inductor and resistance across the input nodes of the amplifier 70.

In response to the toggling of the switch 68, the signal source 66 ceases to generate Iₑ through the inductor 62.

In response to the cessation of Iₑ through the inductor 62, the tracking-point circuit 50 enters an active state, i.e., becomes active. That is, the residual energy stored in the capacitor 54 and in the inductor 58 causes the tracking-point circuit 50 to resonate, or to “ring,” at an underdamped frequency f_{d}=f₀\sqrt{1−ζ²}, where is a damping factor, and

$$ζ = \frac{{R}_{C} + {R}_{L}}{2} \sqrt{\frac{C}{L}} .$$

For example, if R_{C}=0.1 Ohms (Ω), R_{L}=1.0Ω, L=5.6 μH, and C=0.1 μF, then ζ≈0.073 and f_{d}≈99.7% of f₀. Therefore, if R_{C} and R_{L}, and thus are small enough, f_{d}≈f₀.

In response to this ringing, a tracking current I_{L}=Iₜₜ having the frequency f_{d}, and having an exponentially decaying amplitude, flows through the inductor 58 and generates a magnetic field having lines of flux 82.

The flux 82 induces a voltage Vₜ across the series combination of the inductor 62 and the resistance 64.

The voltage Vₜ appears across the input nodes of the amplifier 70, which amplifies Vₜ and generates an AC output voltage Vₒ. Because the resistance across the input nodes of the amplifier 70 is typically high, and therefore, because the current I₆₂ induced by the flux 82 is typically small, in an embodiment I₆₂≈0 while the tracking-point circuit 50 is active during the tracking period Tₜ. To reduce noise on the signal Vₒ, the amplifier 70 can be configured as an integrator or in another suitable topology.

The endovascular imaging system that includes the tracking-point-excitation-and-sensing circuit 52 repeats the above procedure by toggling the switch 68 between the excitation and tracking positions for a suitable amount of time.

As described below, in response to Vₒ during the tracking periods Tₜ while the tracking circuit 50 is active and is operating as a tuned resonator, the endovascular imaging system can determine position and movement information of the location 28 (not shown in FIGS. 9-10) where the tracking point 22 containing the tracking-point circuit 50 is located.

Factors that affect the level of near-field magnetic coupling between the inductors 58 and 62, and therefore affect the magnitudes of the induced current I_{L}=Iₜ during Tₑ and the tracking current I_{L}=Iₜₜ during Tₜ, are the distance D, the difference D_{iff} between the frequencies fₑ and f₀, and the orientation of the inductor 58 relative to the inductor 62. The level of coupling, and therefore, the magnitudes of Iₜ and Iₜₜ, increase as D decreases, D_{iff} decreases, and the orientation is closer to parallel (e.g., the core axis of the inductor 58 is parallel to the core axis of the inductor 62). In contrast, the level of coupling, and therefore, the magnitudes of Iₜ and Iₜₜ, decrease as D increases, D_{iff} increases, and the orientation is further from parallel.

To improve magnetic coupling between the inductors 58 and 62, the endovascular imaging system can reduce the distance D as much as is practical for the application, can sweep the frequency fₑ up and down in a dithering fashion to determine f₀ by equating f₀ with the value of fₑ that delivers the greatest magnitude of Vₒ, and can rotate, or otherwise move, the inductor 62 to determine the orientation relative to the inductor 58 that delivers the greatest magnitude of Vₒ. The endovascular imaging system can perform the latter two procedures at different times, or at the same time.

Still referring to FIGS. 9-10, alternate embodiments are contemplated for the tracking-point circuit 50 and the tracking-point-excitation-and sense circuit 52. For example, one or both of the inductors 58 and 62 may have a respective leakage inductance that does not contribute to the magnetic coupling of the inductors. Furthermore, instead of operating as a tuned resonator, the tracking circuit 50 can include a circuit similar to radio-frequency identification (RFID) circuitry for generating a signal for receiving and sensing by a sense circuit of the endovascular imaging system. Moreover, one or both of the circuits 50 and 52 may include one or more other components in addition to those described, and may have a different topology than described. In addition, the endovascular system can be configured to perform any of the above-described steps, or any other steps, either automatically or with human assistance.

FIG. 11 is a diagram of an excitation coil 90 and of a sensing, or tracking, coil 92, according to an embodiment. For example, the excitation coil 90 can be used as the inductor 62 of FIG. 9 during the excitation period Tₑ (FIG. 10), and the tracking coil 92 can be used as the inductor 62 during the tracking period Tₜ (FIG. 10).

Each of the coils 90 and 92 have a rectangular shape with dimensions w×h, where w and h can be in an approximate range of 10 mm≤w, h≤1000 mm.

The coils 90 and 92 can be disposed in a housing of an endovascular imaging system, and, to increase the level of magnetic coupling with a tracking point 22, can be moveable to adjust their orientations relative to the orientation of the tracking-point inductor 58 (FIG. 9).

Referring to FIGS. 9-11, in operation during an excitation period Tₑ, a switch, such as the switch 68 couples, the coil 90 across the signal source 66 such that the coil 90 generates the excitation flux 80.

And during a tracking period Tₜ, the switch couples the coil 92 across the input nodes of the amplifier 70 such that the coil 92 causes the amplifier 70 to generate Vₒ in response to the tracking flux 82 generated by the inductor 58.

To determine position and movement information in three-dimensional space for a location 28 having three tracking points 22 arranged to generate spatially and electrically orthogonal magnetic fields, the endovascular imaging system can move the coils 90 and 92 in a manner that allows the system to triangulate the location 28 in three-dimensional space. For example, to generate spatially orthogonal magnetic fields, the tracking points 22 can be arranged so that the coil axes of the tracking points are orthogonal to one another (i.e., are aligned with the x, y, and z dimensions of a frame of reference local to the location 28). And to generate electrically orthogonal magnetic fields, the tracking points 22 can be configured, while active, to generate magnetic fields having different frequencies, such as f_{d}, ˜2f_{d}, and ˜3f_{d}.

Referring to FIG. 11, alternate embodiments of the coils 90 and 92 are contemplated. For example, although described as each including a respective single turn, one or both of the coils 90 and 92 can have multiple turns. Furthermore, although described as having rectangular shapes, one or both of the coils 90 and 92 can have a different shape, such as round or oval. Moreover, although described as having the same sizes, one of the coils 90 and 92 can have a different size than the other of the coils. In addition, although described as being active during different periods Tₑ and Tₜ, if the circuits 50 and 52 (FIG. 9) are configured such that the flux 82 is approximately 90° out of phase with the flux 80 (e.g., flux 80 is in phase (I) and flux 82 is quadrature (Q)), then the coils 90 and 92 can be active simultaneously during a continuous combined excitation-and-tracking period Tₑₜ. In addition, one of the coils 90 and 92 can be omitted such that a single coil acts as the inductor 62 (FIG. 9) during both periods Tₑ and Tₜ, or during the combined period Tₑₜ.

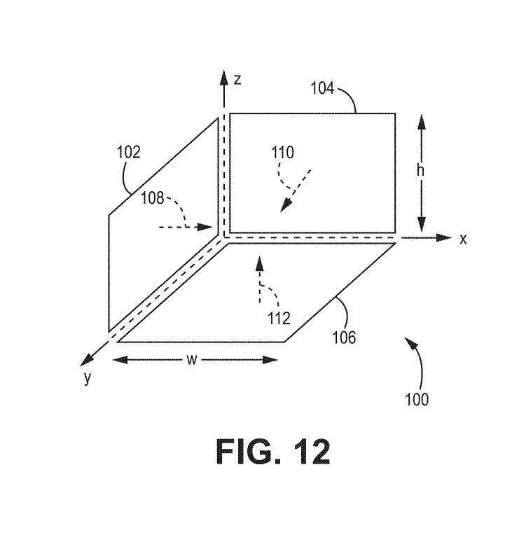

FIG. 12 is a diagram of a coil assembly 100 including spatially orthogonal coils 102, 104, and 106, each of which can be used as the inductor 62 in the tracking-point-exciting circuit 52 of FIG. 9, or as the inductors 62 in respective tracking-point-exciting circuits 52, according to an embodiment.

Each of the coils 102, 104, and 106 has a rectangular shape with dimensions w×h, where w and h can be in an approximate range of 10 mm≤w, h≤1000 mm.

The coil assembly 100 can be disposed in a housing of an endovascular imaging system, and each coil 102, 104, and 106 can be aligned with a respective dimension of a frame of reference of the imaging system. For example, the coil 102 can be in a y-z plane such that its coil axis 108 extends in the x dimension. Similarly, the coil 104 can be in an x-z plane such that its coil axis 110 extends in they dimension, and the coil 106 can be in an x-y plane such that its coil axis 112 extends in the z dimension.

To increase the level of magnetic coupling with spatially and electrically orthogonal tracking points 22 at a location 28 of an endovascular device (not shown in FIG. 12), the coil assembly 100 can be moveable to adjust the magnetic-field orientations of the coils 102, 104, and 106 relative to the magnetic-field orientations of the tracking-point inductors 58 of FIG. 9 (one inductor per tracking point 22).

Referring to FIGS. 9-10 and 12, in operation during an excitation period Tₑ, switches, such as switches 68, couple the coils 102, 104, and 106 across respective signal sources 66 such that the coils each generate respective excitation fluxes 80. The excitation of all of the coils 102, 104, and 106 can be simultaneous if the excitation voltages Vₑ are electrically orthogonal (e.g., have different frequencies) as described above; or, the excitation of the coils can be sequential if the excitation voltages Vₑ are not orthogonal. In the sequential case, the coils 102, 104, and 106 can be switched, sequentially, into a same exciting circuit 52.

And during a tracking period Tₜ, switches couple the coils 102, 104, and 106 across the input nodes of respective amplifiers 70 such that the coils 102, 104, and 106 cause the amplifiers 70 to generate respective voltages Vₒ in response to the respective tracking fluxes 82 generated by the respective inductors 58 of the spatially orthogonal tracking points 22. The tracking using all of the coils 102, 104, and 106 can be simultaneous if the tracking voltages Vᵢₙ are electrically orthogonal (e.g., have different frequencies) as described above; or, the tracking using all of the coils can be sequential if the tracking voltages Vᵢₙ are not orthogonal. In the sequential case, the coils 102, 104, and 106 can be switched sequentially into a same exciting circuit 52.

To determine position and movement information in three-dimensional space for the location 28 having three tracking points 22 arranged to generate spatially and electrically orthogonal magnetic fields, the endovascular imaging system can move the coil assembly 100 in a manner that allows the system to triangulate the location 28 in three-dimensional space. For example, the endovascular imaging system can be moved automatically with one or more motors, or by one or more human operators.

In more detail, each tracking point 22 generates a respective magnetic field that can be measured and mathematically modeled using known techniques and equations.

By moving the tracking coils 102, 104, and 106 to one or more different positions or orientations, a processing circuit (e.g., a microprocessor or a microcontroller) can “fit” one or more curves defined by known equations to the magnetic-field information sensed and provided by the tracking coils.

From these curves, the processing circuit can estimate the locations and orientations of the magnetic fields generated by the tracking points 22 and detected by the tracking coils 102, 104, and 106; and from the estimated magnetic fields, the processing circuit can estimate the locations and orientations of the tracking points in three-dimensional space.

And from the locations and orientations of the tracking points 22, the processing circuit can determine locations and orientations of different points of the device (e.g., lead, catheter) to which the tracking points are attached.

Referring to FIG. 12, alternate embodiments of the coil assembly 100 are contemplated. For example, although described as each including a respective single turn, one or more of the coils 102, 104, and 106 can have multiple turns. Furthermore, although described as having rectangular shapes and the same sizes, one or more of the coils 102, 104, and 106 can have a different shape such as round or oval, or can have a different size as compared to one or more other ones of the coils. Moreover, although described as being active during different periods Tₑ and Tₜ, if the circuits 50 and 52 (FIG. 9) are configured such that the flux 82 is approximately 90° out of phase with the flux 80, then the coils 102, 104, and 106 can be active simultaneously to excite and to track during a continuous combined excitation-and-tracking period Tₑₜ. In addition, each coil 102, 104, and 106 can be associated with a respective corresponding coil (not shown in FIG. 12) such that the coil acts as the inductor 62 (FIG. 9) during one of the periods Tₑ and Tₜ, and the corresponding coil acts as the inductor 62 during the other one of Tₑ and Tₜ, in a manner similar to that described above in conjunction with FIG. 11 for the coils 90 and 92. Furthermore, to reduce the number of times (even down to zero) that the position and orientation of the coil assembly 100 needs to be changed to determine the locations and orientations of the tracking points 22, multiple coil assemblies 100 can be included at different positions and orientations. The number of times that the positions and orientations of each of the multiple coil assemblies 100 can be reduced, even to zero, depends on the number of coil assemblies used; for example, the larger the number of coil assemblies, generally the lower the number of times that the positions and orientations of the multiple coil assemblies 100 need be changed.

Referring to FIGS. 3-12, alternate embodiments of the tracking points 22, circuits 50 and 52, coils 90 and 92, and coil assembly 100 are contemplated. For example, any one or more of these components can be manufactured using semiconductor technology or printed-circuit-board technology. For example, one or more capacitors and coils can be formed on a semiconductor substrate or on a printed circuit board. Furthermore, any alternative described for an embodiment may be applicable to another embodiment.

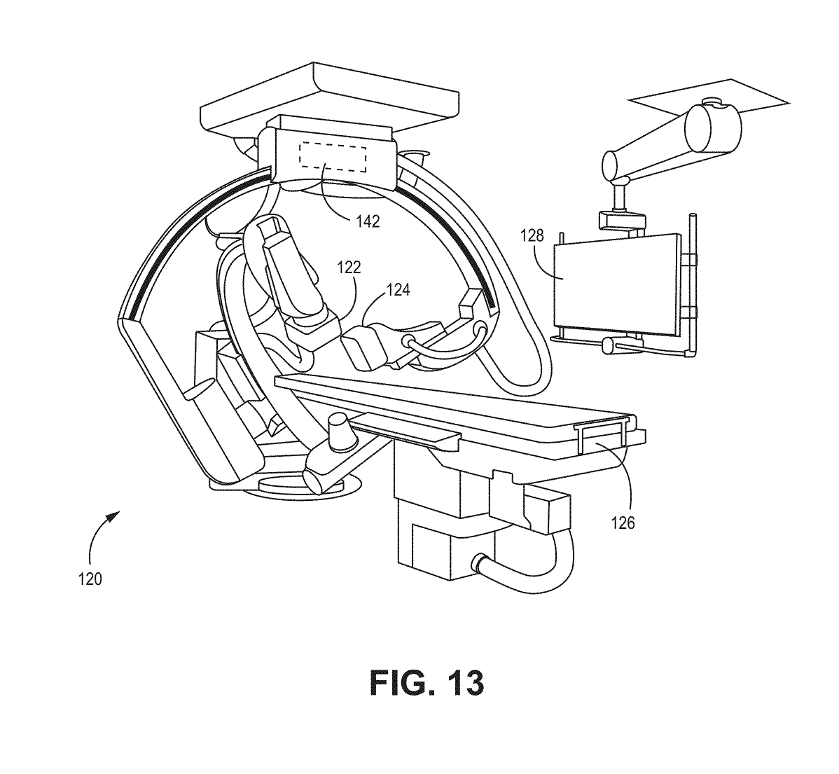

FIG. 13 is a perspective view of an endovascular imaging system 120, according to an embodiment. The system 120 can be similar to a conventional Biplane Fluro Machine, except that the system 120 uses tracking points 22 (FIGS. 3-10) and excitation circuits 52 (FIGS. 9-12) instead of, or in addition to, radiopaque material and radiation to image endovascular devices (e.g., catheters, guide wires) within a body.

The system 120 includes excitation-detection assemblies 122 and 124, which include one or more tracking-point-excitation circuits, such as the circuit 52 of FIG. 9, and one or more sets of coils, such as the coils 90 and 92 of FIG. 11, or one or more coil assemblies, such as the coil assembly 100 of FIG. 12.

In operation of the system 120, a subject (not shown in FIG. 13) lies on a bed 126, and a human operator, or control circuitry of the endovascular imaging system 120, moves the detection assemblies 122 and 124 into positions that allow the one or more excitation circuits and coils or coil assemblies to detect the locations 28 (FIGS. 3-8) of one or more endovascular devices (e.g., catheters, guide wires, stent placers, stent removers) within the body of the subject.

In response to the detected locations 28, computing circuitry of the imaging system 120 can generate, on a display 128, one or more images that include the detected locations such that a doctor can see, at least in a virtual space, the positions of the one or more endovascular devices relative to tissues (e.g., blood vessels) inside of the subject.

Still referring to FIG. 13, alternate embodiments of the endovascular imaging system 120 are contemplated. For example, although the system 120 is described as including two excitation-detection assemblies 122 and 124, the system can include more or fewer than two excitation-detection assemblies.

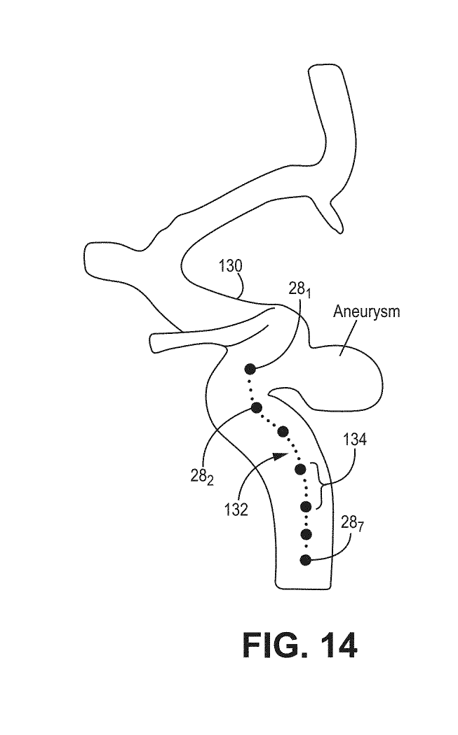

FIG. 14 is an image 130 of a blood vessel inside of a subject's body, where the image includes locations 28₁-28₇ of a guide wire 132 inside of the blood vessel, according to an embodiment. The location 28₁ is at a leading end of the guide wire 132.

The guide wire 132 includes a respective at least one tracking point 22 (FIGS. 3-10) at each location 28, and the imaging device 120 of FIG. 13 can detect the tracking points, and, therefore, can detect the locations, in any manner described above in conjunction with FIGS. 3-13, and then can generate the “dots” in the image 130 to indicate the detected locations 28.

Furthermore, the imaging device 120 of FIG. 13 can generate the image 130 of the blood vessel in a conventional manner. For example, a doctor or other medical professional can inject die into the blood vessel, and the imaging device 120 can irradiate the die-infused blood vessel with x-rays such that the imaging device can “see” the blood vessel. Because the x-rays used to irradiate and image the die-infused blood vessel can be applied while all staff is out of the vicinity of the device 120 (e.g., out of the procedure room), the staff is protected from exposure to the radiation without the need for bulky radiation-protection gear. And because the imaging device 120 effectively images the guide wire 132 with magnetic fields instead of x-rays as described above, the staff is not exposed to radiation while the doctor is inserting the guide wire into, and otherwise performing the procedure on, the subject.

Moreover, the imaging device 120 of FIG. 13 effectively overlays the dots representing the locations 28 onto the appropriate regions of the image 130 of the blood vessel to generate, on the display 128, a virtual representation of the guide wire 132 inside of the blood vessel. That is, image device 120 “fuses” the images of the blood vessel and the location dots into a single image. As described above, such image “fusion” allows imaging the blood vessel separately from the imaging of the guide wire 132.

In addition, the imaging device 120 of FIG. 13 can track movement of the guide wire 132 by tracking the magnetic fields generated by the tracking points 22 as described above in conjunction with FIGS. 3-12, and can update, periodically, the relative positions of the locations 28 within the image 130 to show the new position of the guide wire resulting from any movement, or other change in position, thereof. That is, the imaging device 120 periodically generates new images by infusing the same image of the blood vessel with the updated image of the locations 28.

Still referring to FIG. 14, computing circuitry of the endovascular imaging system 120 (FIG. 13) can interpolate the sections 134 of the guide wire 132 between the locations 28, and, therefore, can generate a virtual representation of the entire guide wire (not of just the locations 28) on the display 128 to further aid the doctor in the procedure that he/she is performing using the guide wire.

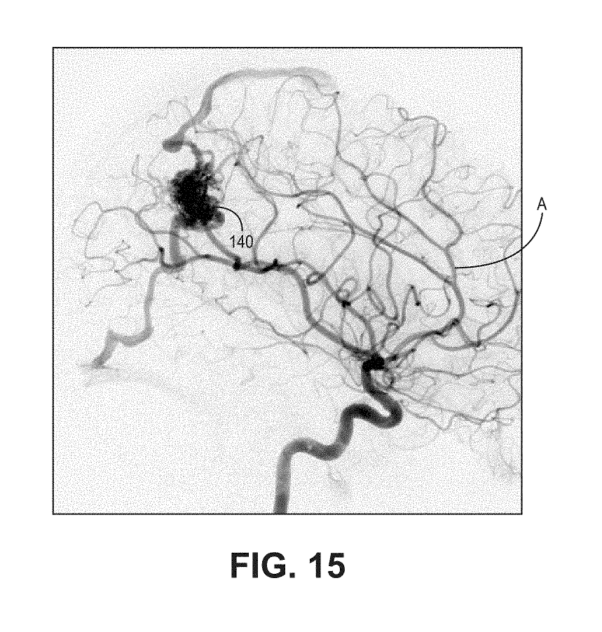

FIG. 15 is an image of an arterial-venous malformation (AVM) 140, and of surrounding blood vessels, according to an embodiment.

In some situations, a doctor may want to move an end of a guide wire inside of one of the blood vessels from an arbitrary point A to the AVM 140, but the doctor doesn't know the best route, or even a suitable route, between point A and the AVM.

Therefore, the doctor may have to try many routes (similar to trying to finding one's way out of a maze) before finding a suitable route.

But, referring to FIG. 13, the endovascular imaging system 120 can include computing circuitry 142 configured to determine one or more suitable routes, even a “best” route, from point A to the AVM 140, much like a driving-direction software application finds one or more suitable routes over roads and highways from one geographic location to another geographic location.

After determining one or more suitable routes, or even a best route, the computing circuitry 142 can, in the image, highlight one or more of these determined routes. For example, the computing circuitry can rank the routes (e.g., with different-colored highlights) from shortest to longest, or by other criteria.

Then, the doctor can select a route from A to the AVM 140, and can move the guide wire to follow the selected route while the imaging system 120 tracks the locations 28 of the guide wire and updates the image to include the locations as described above in conjunction with FIGS. 13-14. In this manner, the doctor can see, via the image on the display 128, whether the guide wire is traversing the selected route.

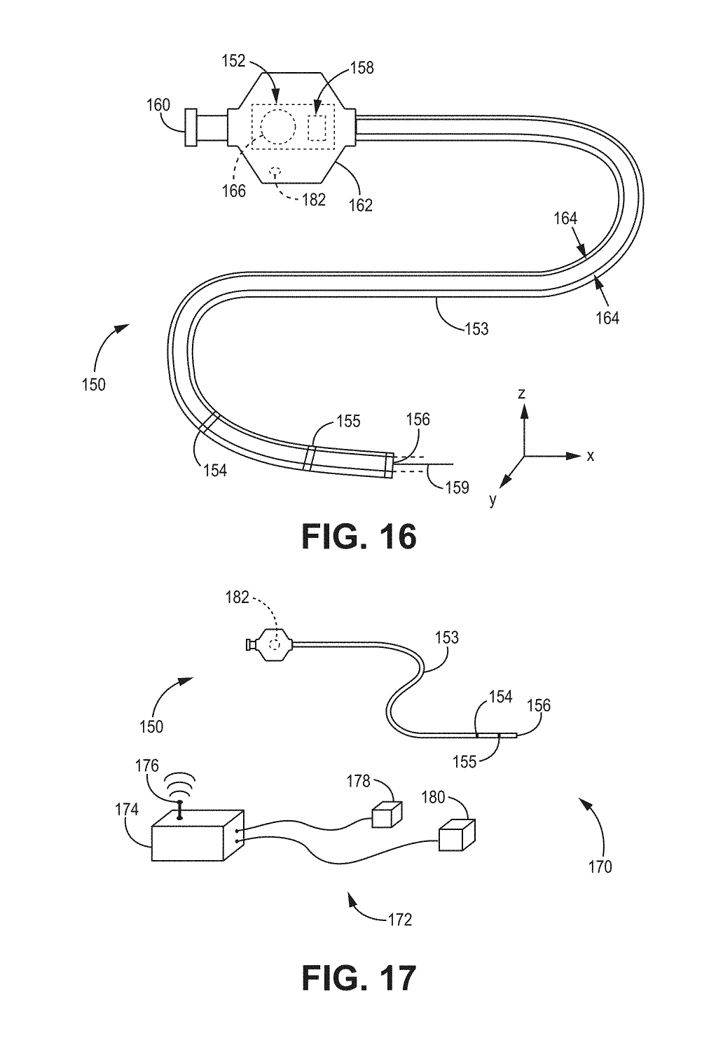

FIG. 16 is a view of an endovascular device 150, which is similar to the endovascular device 20 of FIG. 3 but which includes a power supply 152 and circuitry 158 configured to cause signals generated by one or more electromagnetic antenna coils 154, 155, and 156 to be stronger as compared to the signals generated by the tracking points 22 of FIGS. 3-8, according to an embodiment.

The coils 154, 155, and 156 are wound around an inner or outer surface of a wall of a body 153 of the device 150 such that the magnetic axes of the coils are oriented along an axis 159 of the body. However, other embodiments are possible. For example, the coils 154, 155, and 156 can be oriented such that their magnetic axes are normal to the wall of the body 153, or are at an angle between the axis 159 and orthogonal to the wall of the body. For example, one of the coils 154, 155, and 156 can be configured such that its magnetic axis is oriented approximately along the axis 159, which is an x axis in a coordinate system that is relative to the body 153, and the other coils can be configured such that their magnetic axes are respectively oriented approximately along they and z axes of the same coordinate system. Furthermore, one or more of the coils 154, 155, and 156 can be embedded in the wall of the body 153. Moreover, the spacing between adjacent coils can be any suitable distance along the axis 159, and the spacing between one pair of adjacent ones of the coils 154, 155, and 156 can be approximately the same as, or different from the spacing between another pair of adjacent ones of the coils.

If the coils 154, 156, and 156 are disposed on an outer surface of, or are embedded in, the wall of the body 153, then the cores of the coils include portions of the wall, which can be formed from any material suitable for a catheter. Examples of such materials include metallic compositions such as stainless steel or nitinol, and materials, such as latex, rubber, or plastic, that allow the body 153 flex.

The cores of the coils 154, 155, and 156 also include the hollow interior of the body 153, and any materials disposed therein. While the device 150 is in use, the interior of the body 153 can be filled with fluids such as saline, blood, and medications, and with solids such as a metal guide wire. Because these fluids and solids form at least a respective portion of each of the cores of the coils 154, 155, and 156, these fluids and solids can alter the strengths of the respective magnetic fields that the coils are configured to generate for respective given currents through the coils as compared to the interior of the body 153 being filled with air. To mitigate the affect that materials within the hollow interior of the body 153 have on the magnetic-field strengths of the coil signals, one or more materials (e.g., iron powder, metal strands) of relatively high magnetic permeability can be disposed on one or more of the inner and outer surfaces of, or can be embedded within, the walls of the body in a manner that preserves the flexibility of the body. Increasing the permeability of the cores of the coils 154, 155, and 156 also can provide a benefit of increasing the strengths of the magnetic fields that the coils respectively generate.

Still referring to FIG. 16, the device 150 further includes a lumen access point 160, through which tools and other items, such as a guide wire and a stent, can enter the device.

The device 150 still further includes, near the lumen access point 160, a housing 162, in which are disposed the power supply 152 and the other circuitry 158, which is configured to drive, electrically, the coils 154, 155, and 156, and to receive respective signals from the coils. For example, a respective pair of conductive leads 164 (only one pair shown in FIG. 16) couples the coil 156 to the circuitry 162; a respective similar pair of conductive leads (not shown in FIG. 16) couples each of the other coils to the circuitry 162. The conductive leads 164 can be embedded within the wall of the body 153, can be disposed on an inner or outer surface of the wall and left exposed, or can be disposed between an inner or outer surface of the wall and a protective sheathing of a non-conductive material such as the same material from which the body 153 is formed. Furthermore, the power supply 152 can be configured to receive and to hold one or more batteries 166, and both the power supply and the other circuitry 158 each can include one or more respective integrated circuits.

FIG. 17 is a perspective view of an endovascular-device-and-tracking system 170, which includes the endovascular device 150 of FIG. 16 and a tracking subsystem 172, according to an embodiment.

The tracking subsystem 172 includes a console 174, an antenna, such as a high-frequency (HF) antenna, 176, and one or more (two shown in FIG. 17) three-axis antenna assemblies 178 and 180, which can be, for example, low-frequency (LF) three-axis antenna assemblies.

The console 174 includes a power supply and circuitry, such as computing circuitry (e.g., a microcontroller or microprocessor), configured to track the body 153, particularly the coils 154, 155, and 156, of the endovascular device 150 while the body is inside of a subject (e.g., inside of an artery of a subject). The console 174 is configured to receive, via the antennas 178 and 180, location electromagnetic signals generated by the coils 154, 155, and 156, and, in an embodiment in which the endovascular device 150 lacks a power supply, the console 174 is configured to excite the coils 154, 155, and 156 via the antennas 178 and 180, for example, as described above in conjunction with FIGS. 9-10.

The console 174 is also configured to communicate with the circuitry 158 (FIG. 16) onboard the endovascular device 150 via the antenna 176 and an antenna 182, such as an HF antenna, onboard the endovascular device. For example, the console 174 can send, to the circuitry 158, commands that configure the endovascular device 150 for a particular type of operation, that request status information from the endovascular device, and that can cause the endovascular device to transmit signals via the coils 154, 155, and 156 at a particular time, or according to a particular timing pattern, so that the console “knows” when to expect that the antennas 178 and 180 will receive one or more signals from the coils 154, 155, and 156. The console 174, for example, can send, to the endovascular device 150, commands that cause the circuitry 158 to transmit a signal from a particular coil 154, 155, and 156 at a particular time so that the circuitry 158 “knows” which coil is transmitting a signal, and, therefore, can determine the location of that particular coil more easily than if all of the coils 154, 155, and 156 were transmitting simultaneously.

The antenna assemblies 178 and 180 can be similar to the antenna assembly 100 of FIG. 12, and can include antennas similar to the antennas 90 and 92 of FIG. 9 or antennas 102, 104, and 106 of FIG. 12.

Still referring to FIG. 17, alternate embodiments of the system 170 are contemplated. For example, the subsystem 170 can include multiple endovascular devices 150, or multiple tracking subsystems 172. Furthermore, the tracking subsystem 172 can form part (e.g., a subsystem) of an imaging device, such as the imaging device 120 of FIG. 13. In addition, the tracking console 174 and the electronics unit 152 can include high-frequency antennas 176 and 182 to facilitate radio-frequency communication between the tracking console and the electronics unit 152 via a wireless communication link. This allows the tracking console 174 and the endovascular device 150 to exchange system information such as timing, status, and signal data. The data sent by the electronics unit 152 through the high-frequency communication link can aid the tracking console 174 in determining the location of various areas of the endovascular device as it moves through, e.g., a blood vessel.

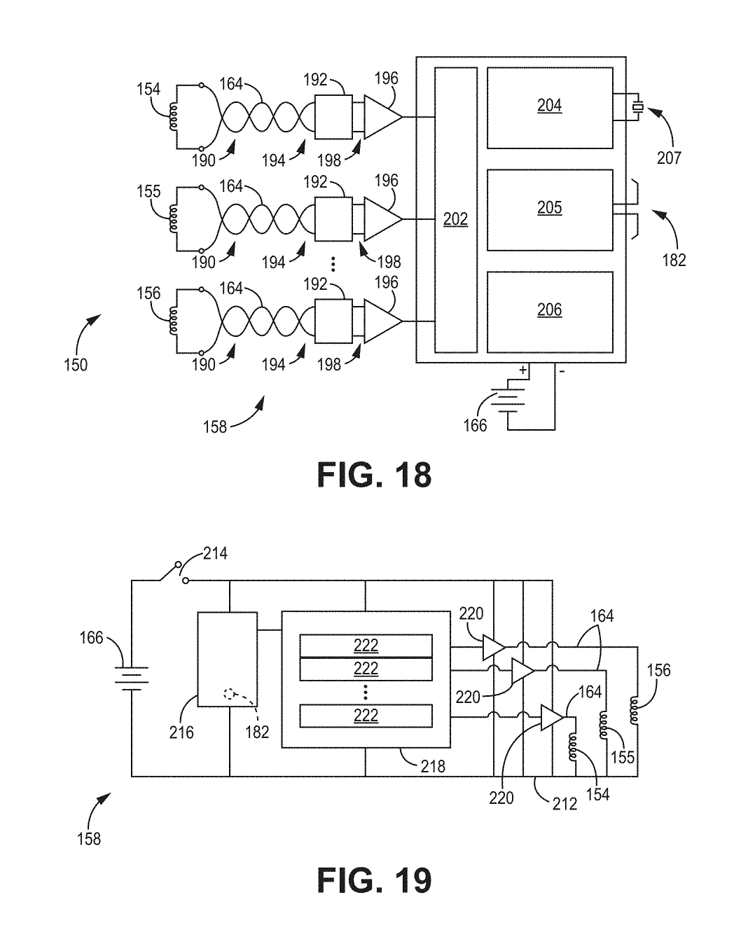

FIG. 18 is a diagram of the coils 154, 155, and 156, the leads 164, and at least a portion the circuitry 158, of the endovascular device 150 of FIG. 16, according to an embodiment.

The leads 164 are arranged in twisted pairs 190, one twisted pair of leads per each coil 154, 155, and 156. Twisting each pair 190 of leads 164 effectively shields the leads by making them less sensitive to noise and other extraneous signals that the leads may otherwise “pick up,” and, therefore, that may otherwise interfere with, the signals received by the respective one of the coils 154, 155, and 156. Electromagnetic shielding (e.g., a conductive material) can also be included on one or more walls of the body 153 (FIGS. 16-17) of the endovascular device 150.

The circuitry 158 includes impedance-matched filter circuits 192, one filter circuit per twisted pair 190 of leads 164. The filter circuits 192 can be bandpass filters that are configured to pass the signals received by the coils 155, 156, and 157, respectively, and to reject other signals (e.g., noise) at frequencies other than the frequencies of the signals received by the coils. Each filter circuit 192 includes a respective differential input port 194, which presents to the respective twisted pair 190 an input impedance that approximately matches the characteristic impedance of the twisted pair so as to provide improved transfer of signal power from the twisted pair to the filter circuit (a respective circuit configured for similar impedance matching also can be disposed between each coil 154, 155, and 156 and the respective twisted pair 190).

Furthermore, the circuitry 158 includes differential-input-single-ended-output amplifiers 196, each coupled to receive a filtered coil signal from a respective one of the filter circuits 192. Having a respective differential input 198 allows each amplifier 196 to reject noise and other signals at frequencies outside of the frequency range of the respective filtered coil signal, which is the signal of interest.

The circuitry 158 also includes one or more Analog-to-Digital Converters (ADCs) 202 (one three-channel ADC shown in FIG. 18). Each channel of the ADC 202 is configured to convert a signal from a respective one of the amplifiers 196 from an analog signal to a corresponding digital signal. For example, the topmost channel of the ADC 202 is configured to receive the analog signal output by the topmost amplifier 196, and to convert this analog signal into a corresponding binary digital signal. If the ADC 202 is configured to have a sampling rate that is high enough (e.g., at least eight times the frequency of the signals received by the coils 154, 155, and 156), then the ADCs can generate respective digital signals that represent the amplitudes and phases of the amplified and ranged analog signals on a cycle-by-cycle basis. Or, to reduce cost and complexity, the circuitry 158 can include, instead of the ADC 202, rectification and envelope-detection circuitry to determine the amplitudes of the signals from the coils 154, 155, and 156 without phase information.

In addition, the circuitry 158 includes a timing circuit 204, a radio circuit 205, and a power-management circuit 206.

The timing circuit 204 is configured to generate one or more clock signals in response to a master signal having a frequency that a component, such as a crystal 207, is configured to set. The ADC 202, and possibly the radio circuit 205, the power-management circuit 206, and other components or sections (e.g., digital components or sections) of the circuitry 158, use the one or more clock signals for timing and other functions.

The radio circuit 205 includes the antenna 182, and is configured to communicate with the tracking console 174 (FIG. 17) via the antennas 176 (FIG. 17) and 208. For example, the radio circuit 205 is configured to transmit, to the tracking console 174, the one or more digitized amplifier-output signals (three signals shown in, and described in conjunction with, FIG. 18) from the amplifiers 196. The radio circuit 205 also can be configured to receive commands, requests, configuration data, or program instructions from the tracking console 174, and to send information, such as status information, to the tracking console in response to a request for the information. The radio circuit 205 and the antenna 182 can be configured for communications over any suitable frequency band.

And the power-management circuit 206 generates, in response to the battery 166, one or more power-supply signals (e.g., voltages or currents) for components and sections of the circuitry 158. For example, the power-management circuit 206 can be configured to generate one or more respective supply signals for each of the filter circuits 192 (if the filter circuits are active filter circuits), for each of the amplifiers 196, for the ADC 202, for the timing circuit 204, and for the radio circuit 206. The power-management circuit 206 also can be configured to charge the battery 166 from an external power source to which the power-management circuit is coupled via a connector (not shown in FIG. 18) or wirelessly.

Still referring to FIG. 18, alternate embodiments of the circuitry 158 is contemplated. For example, one or more of the filter circuits 192 can be omitted from the circuitry 158. Furthermore, one or more other components can be omitted from the circuitry 158, and one or more components not shown or described can be added to the circuitry 158. In addition, the circuitry 158 can include computing circuitry that is configured to determine, from the digitized coil signals generated by the ADC 202, the relative positions and orientations of the coils 154, 155, and 156 from the coil signals, and the radio circuit 205 can be configured to transmit the relative positions and orientations of the coils to the tracking console 174 of FIG. 17. Or, the circuitry 158 can be configured to transmit, via the high-frequency antenna 182 (FIG. 17), information (e.g., amplitude, phase) regarding the digitized coil signals to the console 174, which can include circuitry configured to determine the relative positions and orientations of the coils 154, 155, and 156 from this information.

FIG. 19 is a diagram of the coils 154, 155, and 156, the leads 164, and at least a portion the circuitry 158, of the endovascular device 150 of FIG. 16, according to another embodiment in which the circuitry 158 is configured to generate signals that the coils 154, 155, and 156 transmit.

The leads 164 are arranged as single leads, one lead per coil 154, 155, and 156, and a ground/return lead 212; alternatively, each of one or more of the leads 164 is a twisted pair such as described above in conjunction with FIG. 18. Electromagnetic shielding (e.g., a conductive material) can also be included on one or more walls of the body 153 (FIGS. 16-17) of the endovascular device 120.

In addition to the one or more batteries 166, the circuitry 158 includes a switch 214, a radio 216, a programmable controller 218 (e.g., a microprocessor, microcontroller, field-programmable gate array (FPGA)), and amplifiers 220 (one per coil 154, 155, and 156).

The switch 214 is configured to couple and to uncouple the battery(ies) 166, or other power source, to and from the rest of the circuitry 158. The switch 214 can be a mechanical switch, an electronic switch, or any other suitable type of switch.

The radio 216 is configured to send and to receive communications to and from the console 174 of the tracking subsystem 172 (FIG. 17) via the antennas 182 and 176 (FIG. 17). The radio 216 can be, or can include, any suitable type of transmit and receive circuitry, and can be similar to, or the same as, the radio circuit 205 of FIG. 18.

The programmable controller 218 includes a respective waveform generator 222 for each coil 154, 155, and 156. Each waveform generator 222 is configurable, with software, firmware, or another bit set (hereinafter “waveform data”), to generate a signal having characteristics, such as amplitude, phase, wave shape, and time duration, corresponding to the respective waveform data. The programmable controller 218 is configured to receive the waveform data for each waveform generator 222 from the tracking console 174, or from another source, via the radio circuit 216.

And each of the amplifiers 220 is configured to amplify a signal from a respective waveform generator 222, and to drive a respective one of the coils 154, 155, and 156 with the amplified signal.

And each coil 154, 155, and 156 is configured to generate a respective magnetic field in response to the amplified signal from a respective one of the amplifiers 220.

For example, in operation, the programmable controller 218 can cause the waveform generators 222 to generate signals that are electrically orthogonal to one another so that the tracking subsystem 172 (FIG. 17) can distinguish between the signals, and determine from which coil 154, 155, and 156 each signal originated. Being able to distinguish which signal came from which coil facilitates the circuitry of the console 174 (FIG. 17) determining the respective position and the respective orientation of each coil, and, therefore, facilitates the circuitry of the console determining a position and an orientation of the body 153 of the endovascular device 150 (FIGS. 16-17).

In an alternative operation, the programmable controller 218 can cause the waveform generators 222 to generate signals having similar characteristics, and the console circuitry can determine a relative position and a relative orientation of the body 153 of the endovascular device 150 based on a respective amplitude and a respective relative phase of each of the signals that the console circuitry receives from the coils 154, 155, and 156.

Still referring to FIG. 19, alternate embodiments of the circuitry 158 are contemplated. For example, one or more components can be omitted from the circuitry 158, and one or more components not shown or described can be added to the circuitry 158. Furthermore, the circuitry 158 can include a port configured to receive, from an external source programming or configuration data for configuring the programmable controller 210. In addition, the circuitry 158 of FIG. 19 can be combined with the circuitry 158 of FIG. 18 on a single endovascular device such as the endovascular device 150 of FIGS. 16-17.

Example 1 includes a wireless tracking point.

Example 2 includes a wireless tracking point configured to resonate and to generate an oscillating magnetic field in response to an excitation signal.

Example 3 includes an imaging device configured to fuse an image of a location of a wireless tracking point with an image of a body part.

Example 4 includes an imaging device having one or more tracking-excitation coils.

Example 5 includes an imaging device having one or more tracking coils.

Example 6 includes an imaging device having one or more excitation coils.

Example 7 includes an imaging device configured to determine a route between one location within a body and another location within the body.

Example 8 includes a method comprising determining a location on an object within a body by exciting and detecting a magnetic field generated by a tracking point at the location.

Example 9 includes a computer-readable medium storing software instructions, that when executed by computing circuitry, causes the circuitry to generate an image showing locations determined in response to a magnetic field detected by respective tracking points at the locations.

Example 10 includes a wireless tracking endovascular device.

Example 11 includes an apparatus, comprising: a first tracking unit configured for mounting to an endovascular device and to generate a first magnetic field along a first dimension; and a second tracking unit configured for mounting to the endovascular device and to generate a second magnetic field along a second dimension that is approximately orthogonal to the first dimension.

Example 12 includes the apparatus of Example 11 wherein: the first tracking unit includes a first coil configured to generate the first magnetic field; and the second tracking unit includes a second coil configured to generate the second magnetic field.

Example 13 includes the apparatus of any of Examples 11-12 wherein: the first tracking unit includes a first resonant circuit configured to generate the first magnetic field by resonating at a first frequency; and the second tracking unit includes a second resonant circuit configured to generate the second magnetic field by resonating at a second frequency.

Example 14 includes an endovascular device, comprising: a body configured for insertion into a body of a lifeform; a first tracking unit disposed at a first location of the body and including a first coil configured to generate a first magnetic field; and a second tracking unit disposed at a second location of the body and including a second coil configured to generate a second magnetic field.

Example 15 includes the endovascular device of Example 14, wherein: the first tracking unit is configured to generate the first magnetic field approximately parallel to a first axis of the body; and the second tracking unit is configured to generate the second magnetic field approximately parallel to a second axis of the body, the second axis being orthogonal to the first axis.

Example 16 includes the endovascular device of any of Examples 14-15, wherein: the first tracking unit is configured to receive a first excitation signal wirelessly from an external source, and to generate the first magnetic field approximately parallel to a first axis of the body in response to the first excitation signal; and the second tracking unit is configured to receive a second excitation signal wirelessly from an external source, and to generate the second magnetic field approximately parallel to a second axis of the body in response to the second excitation signal, the second axis being orthogonal to the first axis.

Example 17 includes the endovascular device of any of Examples 14-16, further comprising: a controller circuit configured to generate first and second drive signals that are approximately electrically orthogonal to one another; wherein the first tracking unit is configured to generate the first magnetic field in response to the first drive signal; and wherein the second tracking unit is configured to generate the second magnetic field in response to the second drive signal.

Example 18 includes the endovascular device of any of Examples 14-17, further comprising: a controller circuit configured to generate first and second drive signals that are approximately electrically orthogonal to one another; wherein the first tracking unit is configured to generate, in response to the first drive signal, the first magnetic field approximately parallel to a first axis of the body; and wherein the second tracking unit is configured to generate, in response to the second drive signal, the second magnetic field approximately parallel to a second axis of the body, the second axis being orthogonal to the first.

Example 19 includes the endovascular device of any of Examples 14-18, further comprising: a controller circuit configured to generate first and second drive signals; wherein the first tracking unit is configured to generate, in response to the first drive signal, the first magnetic field approximately parallel to a first axis of the body; and wherein the second tracking unit is configured to generate, in response to the second drive signal, the second magnetic field approximately parallel to a second axis of the body, the second axis being orthogonal to the first axis.

Example 20 includes the endovascular device of any of Examples 14-19, further comprising: a controller circuit configured to generate first and second drive signals; wherein the first tracking unit is configured to generate the first magnetic field in response to the first drive signal; and wherein the second tracking unit is configured to generate the second magnetic field in response to the second drive signal.

Example 21 includes the endovascular device of any of Examples 14-20, further comprising: a controller circuit configured to generate first and second drive signals; a power supply configured to power the controller circuit; wherein the first tracking unit is configured to generate the first magnetic field in response to the first drive signal; and wherein the second tracking unit is configured to generate the second magnetic field in response to the second drive signal.

Example 22 includes the endovascular device of any of Examples 14-21, further comprising: a controller circuit configured to generate first and second drive signals; a power supply including a battery and configured to power the controller circuit; wherein the first tracking unit is configured to generate the first magnetic field in response to the first drive signal; and wherein the second tracking unit is configured to generate the second magnetic field in response to the second drive signal.

Example 23 includes the endovascular device of any of Examples 14-22, further comprising a third tracking unit disposed at a third location of the body and including a third coil configured to generate a third magnetic field.

Example 24 includes an endovascular device, comprising: a body configured for insertion into a lifeform; a first tracking unit disposed at a first location of the body and including a first coil configured to generate a first signal in response to a first magnetic field, the first signal related to the first location; and a second tracking unit disposed at a second location of the body and including a second coil configured to generate a second signal in response to a second magnetic field, the second signal related to the second location.

Example 25 includes the endovascular device of Example 24, further comprising a transmitter configured to send, to a location remote from the body, information related to the first and second signals.

Example 26 includes the endovascular device of any of Examples 24-25, further comprising circuitry configured: to determine the first location in response to the first signal; to determine the second location in response to the second signal; and to send information representative of the first and second locations to a location remote from the body.

Example 27 includes an endovascular-device tracker, comprising: at least one antenna configured to generate a signal in response to a magnetic field; and circuitry configured to determine, in response to the signal, a location of a source of the magnetic field, the location being inside of a lifeform.

Example 28 includes the endovascular-device tracker of Example 27 wherein the circuitry is configured to determine, in response to the signal, an orientation of the source of the magnetic field.

Example 29 includes the endovascular-device tracker of any of Examples 27-28 wherein an orientation of an aperture of the antenna is movable.

Example 30 includes the endovascular-device tracker of any of Examples 27-29, further comprising: wherein the circuitry is configured to generate data representing an image of an internal section of the lifeform and an indication of the location within the internal section; and a display configured to render the image in response to the data.

Example 31 includes an endovascular-device tracker, comprising: at least one antenna configured to excite a coil of an endovascular device while the coil is inside of a lifeform; a receiver configured to receive, from the endovascular device, a first signal that is related to a second signal generated by the excited coil; and circuitry configured to determine, in response to the first signal, a location of the coil.

Example 32 includes the endovascular-device tracker of Example 31 wherein the circuitry is configured to determine, in response to the first signal, an orientation of the coil.

Example 33 includes the endovascular-device tracker of any of Examples 31-32 wherein: an orientation of an aperture of at least one of the at least one antenna is movable; and the circuitry is configured to determine, in response to the first signal and to the orientation of the aperture of at least one of the at least one antenna, a location of the coil.

Example 34 includes a system, comprising: an endovascular device including a body, and a magnetic element disposed at a location of the body and configured to generate a first signal from which the location can be determined while the body is disposed inside of the lifeform; and an endovascular-device tracker including at least one antenna configured to receive, from the endovascular device, a second signal that is related to the first signal; and circuitry configured to determine, in response to the second signal, the location of the body of the endovascular device.

Example 35 includes the system of Example 34 wherein the magnetic element includes a conductive winding.

Example 36 includes a method, comprising: inserting a portion of an endovascular device into a body of a lifeform; and generating each of one or more magnetic fields at a respective one of one or more locations of the portion of the of the endovascular device.

Example 37 includes a method, comprising: inserting a portion of an endovascular device into a body of a lifeform; and generating each of one or more signals in response to a respective one of one or more magnetic fields sensed at a respective one of one or more locations of the portion of the endovascular device, each of the one or more signals carrying information related to the respective one of the one or more locations.

Example 38 includes a method, comprising: sensing a magnetic field; and determining, in response to the magnetic field, a location of a source of the magnetic field, the location being inside of a body of a lifeform.

Example 39 includes a method, comprising: exciting a magnetic-field sensor of an endovascular device while the sensor is inside a body of a lifeform; receiving, from the endovascular device, a first signal that is related to a second signal generated by the excited magnetic-field sensor; and determining an approximate location of the magnetic-field sensor in response to the first signal.

Example 40 includes a computer-readable medium storing software instructions, that when executed by computing circuitry, causes the circuitry: to sense a magnetic field; and to determine, in response to the magnetic field, a location of a source of the magnetic field, the location being inside of a body of a lifeform.

Example 41 includes a computer-readable medium storing software instructions, that when executed by computing circuitry, causes the circuitry: to excite a magnetic-field sensor of an endovascular device while the sensor is inside a body of a lifeform; to receive, from the endovascular device, a first signal that is related to a second signal generated by the excited magnetic-field sensor; and to determine an approximate location of the magnetic-field sensor in response to the first signal.

From the foregoing it will be appreciated that, although specific embodiments have been described herein for purposes of illustration, various modifications may be made without deviating from the spirit and scope of the disclosure. Furthermore, where an alternative is disclosed for a particular embodiment, this alternative may also apply to other embodiments even if not specifically stated. Moreover, one or more components of a described apparatus or system may have been omitted from the description for clarity or another reason. In addition, one or more components of a described apparatus or system that have been included in the description may be omitted from the apparatus or system. Furthermore, one or more steps of a described procedure may have been omitted from the description for clarity or another reason. And one or more steps of a described procedure that have been included in the description may be omitted from the procedure.

1.-3. (canceled)

4. An endovascular device, comprising:
a body configured for insertion into a body of a lifeform;
a first tracking unit disposed at a first location of the body and including a first coil configured to generate a first magnetic field; and
a second tracking unit disposed at a second location of the body and including a second coil configured to generate a second magnetic field.

5. The endovascular device of claim 4, wherein:
the first tracking unit is configured to generate the first magnetic field approximately parallel to a first axis of the body; and
the second tracking unit is configured to generate the second magnetic field approximately parallel to a second axis of the body, the second axis being orthogonal to the first axis.

6. The endovascular device of claim 4, wherein:
the first tracking unit is configured
to receive a first excitation signal wirelessly from an external source, and
to generate the first magnetic field approximately parallel to a first axis of the body in response to the first excitation signal; and
the second tracking unit is configured
to receive a second excitation signal wirelessly from an external source, and
to generate the second magnetic field approximately parallel to a second axis of the body in response to the second excitation signal, the second axis being orthogonal to the first axis.

7. The endovascular device of claim 4, further comprising:
a controller circuit configured to generate first and second drive signals that are approximately electrically orthogonal to one another;
wherein the first tracking unit is configured to generate the first magnetic field in response to the first drive signal; and
wherein the second tracking unit is configured to generate the second magnetic field in response to the second drive signal.

8. The endovascular device of claim 4, further comprising:
a controller circuit configured to generate first and second drive signals that are approximately electrically orthogonal to one another;
wherein the first tracking unit is configured to generate, in response to the first drive signal, the first magnetic field approximately parallel to a first axis of the body; and
wherein the second tracking unit is configured to generate, in response to the second drive signal, the second magnetic field approximately parallel to a second axis of the body, the second axis being orthogonal to the first axis.

9. The endovascular device of claim 4, further comprising:
a controller circuit configured to generate first and second drive signals;
wherein the first tracking unit is configured to generate, in response to the first drive signal, the first magnetic field approximately parallel to a first axis of the body; and
wherein the second tracking unit is configured to generate, in response to the second drive signal, the second magnetic field approximately parallel to a second axis of the body, the second axis being orthogonal to the first axis.

10. The endovascular device of claim 4, further comprising:
a controller circuit configured to generate first and second drive signals;
wherein the first tracking unit is configured to generate the first magnetic field in response to the first drive signal; and
wherein the second tracking unit is configured to generate the second magnetic field in response to the second drive signal.

11. The endovascular device of claim 4, further comprising:
a controller circuit configured to generate first and second drive signals;
a power supply configured to power the controller circuit;
wherein the first tracking unit is configured to generate the first magnetic field in response to the first drive signal; and
wherein the second tracking unit is configured to generate the second magnetic field in response to the second drive signal.

12. The endovascular device of claim 4, further comprising:
a controller circuit configured to generate first and second drive signals;
a power supply including a battery and configured to power the controller circuit;
wherein the first tracking unit is configured to generate the first magnetic field in response to the first drive signal; and
wherein the second tracking unit is configured to generate the second magnetic field in response to the second drive signal.

13. The endovascular device of claim 4, further comprising a third tracking unit disposed at a third location of the body and including a third coil configured to generate a third magnetic field.

14.-16. (canceled)

17. An endovascular-device tracker, comprising:
at least one antenna configured to generate a signal in response to a magnetic field; and
circuitry configured to determine, in response to the signal, a location of a source of the magnetic field, the location being inside of a lifeform.

18. The endovascular-device tracker of claim 17 wherein the circuitry is configured to determine, in response to the signal, an orientation of the source of the magnetic field.

19. The endovascular-device tracker of claim 17 wherein an orientation of an aperture of the antenna is movable.

20. The endovascular-device tracker of claim 17, further comprising:
wherein the circuitry is configured to generate data representing an image of an internal section of the lifeform and an indication of the location within the internal section; and
a display configured to render the image in response to the data.

21.-27. (canceled)

28. A method, comprising:
sensing a magnetic field; and
determining, in response to the magnetic field, a location of a source of the magnetic field, the location being inside of a body of a lifeform.

29.-31. (canceled)
